(19) United States
(12) Patent Application Publication
(10) Pub. No.: US 2012/0122694 A1
(43) Pub. Date: May 17, 2012

(54) VERBESSERTE MICROKAPSELN UND IHRE HERSTELLUNG IMPROVED MICROCAPSULES, AND THEIR PRODUCTION THEREOF

(75) Inventors: Klaus Last, Osterode (DE); Daniel Mues, leese (DE)

(73) Assignee: Follmann & Co. Gesellschaft fur Chemie-Werkstoffe und Verfahrenstechnik mbH & Co. KG, Minden (DE)

(21) Appl. No.: 13/255,693

(22) PCT Filed: Mar. 12, 2010
(86) PCT No.: PCT/EP2010/001572

§ 371 Date: Dec. 20, 2011

(30) Foreign Application Priority Data
Mar. 12, 2009 (DE) 1020090124551

(51) Int. Cl. C08F 228/02; A61K 8/11; A01N 25/28; C09D 141/00; A01P 7/04; B01J 31/06; A01P 3/00; A01P 1/00; C09K 5/00; C09K 3/00; C10M 129/68; A61K 9/50; A01P 13/00

(52) U.S. Cl. 504/359; 526/287; 521/149; 521/136; 424/489; 424/401; 512/4; 252/79; 252/364; 508/405; 502/159; 428/402

(57) ABSTRACT

The invention refers to microcapsules whose capsule wall comprise resin, and which results through reaction of at least one aromatic alcohol or its ether or derivative and at least one aldehyde component, which includes at least two C-atoms per molecule.

The invention refers to microcapsules having walls comprised of resin and which results from the reaction of at least one alcohol with at least an aldehyde component that includes at least two C-atoms per molecule, as well as dispersions that contain such microcapsules. In addition, subject matter of the invention includes the use and the production of microcapsules/microcapsule dispersions and products that contain such microcapsules/microcapsule dispersions and their use. A further subject matter of the present invention are new AMPS-copolymers, which are suitable as protective colloids, for example, in the production of microcapsules.

From the prior art, microcapsules are known that can contain as core material liquid, solid or gaseous material. Normally used as material for capsule walls are for example, phenol-formaldehyde-polymers, melamine-formaldehyde-polymers, polyurethane, gelatin, polyamide or polyurea. Widely used are for example leuko dye-filled microcapsules for the production of carbonless papers.

From U.S. Pat. No. 3,755,190 it is known that capsules from phenol-formaldehyde-polymer have brittle walls. In order to avoid this, a method of production is described whereby completely hydrolyzed polyvinyl alcohol is utilized.

Dispersions of microcapsules from aminoplast resins, such as melamine-formaldehyde resins contain, depending on production conditions, a certain portion of free formaldehydes. Due to concerns about the environment and work environment hygiene, it is desirable to keep the formaldehyde content as low as possible, and if possible, to avoid it altogether. To reduce the formaldehyde content usually formaldehyde scavengers are added to microcapsule dispersions of melamine-formaldehyde-resins. The formaldehyde scavengers used most often are ammonia, urea, ethylene urea and melamine that reduce the residual content of formaldehyde in the capsule dispersion.

From EP-A 0383 358 and DE-A 38 14 250 light sensitive materials are known that consist of microcapsules whose walls are formed from melamine-formaldehyde-resin. To remove the residual formaldehyde, urea is used during hardening.

In the methods as described in EP-A 319 337 and U.S. Pat. No. 4,918,317, urea is used at the end of hardening.

EP-A 0415 273 describes the production and use of mono- and poly-dispersed full sphere particles from melamine-formaldehyde-condensate. For binding the formaldehyde that is released during condensation, the use of ammonia, urea or ethylene urea is proposed.

Microcapsules from melamine-formaldehyde-resins that are produced by utilizing sulfonic acid-groups-containing polymers are marked by their uniform capsule size and consistency (EP-A 0218 887 and EP-A 0 026 914). These capsule dispersions contain however residual free aldehyde that is undesirable for further processing.

Thus, EP-A 0 026 914 recommends to bind the formaldehyde following the hardening with ethylene urea and/or utilize melamine as a formaldehyde scavenger.

From the DE 198 35 114, dispersions of microcapsules are known on the basis of melamine-formaldehyde-resin, whereby the melamine-formaldehyde-resin is partially etherified and contains water soluble primary, secondary or tertiary amine or ammonia. Before hardening, the formaldehyde scavenger is added.

DE 198 33 347 describes a process for the production of microcapsules through condensation of melamine-formaldehyde-resins and/or their methyl ethers, wherein before the hardening, urea or urea as formaldehyde scavenger whose amino groups are coupled with an ethylene or propylene group are added. The resulting dispersions, while low on aldehyde, the stability of the microcapsules and the viscosity of the microcapsule dispersion are however impacted in a negative way.

WO 01/51197 teaches a process for the production of microcapsules through condensation of melamine-formaldehyde-resins, wherein during hardening a mixture from melamine and urea is added.

Through addition of the named aldehyde scavengers to the completed microcapsule dispersion or during production of the microcapsule, the formaldehyde content of the microcapsule dispersion is being routinely lowered. However, in many cases the formaldehyde content of products that contain microcapsule dispersions or that are treated with them, cannot be reduced below a certain level even when large quantities of formaldehyde scavenger have been added.

Thus, an object of the present invention is to develop microcapsules having a low formaldehyde content or preferably to avoid use of formaldehyde entirely.

These objects are solved by the microcapsules according to the present invention, whose walls include a resin and which results from the reaction of:

a) at least one alcohol or its ether or derivatives with

b) at least one aldehyde component that includes at least two C-atoms per molecule, and

c) optionally at least one (meth)acrylate-polymer.

The present invention refers also to microcapsule dispersions that contain such microcapsules according to the invention.

In addition, the present invention provides a process for the production of microcapsules according to the invention and microcapsule dispersion where a) the at least one alcohol (or its ether or derivatives) is mixed and reacted with b) at least an aldehyde component that includes at least two C-atoms per molecule, and c) optionally with at least one (meth)acrylate-polymer, and wherein the capsules are later hardened.

Within the framework of the present invention, the preferred aromatic alcohols are aryloxyalkanols, arylalkanols and oligoalkanolarylethers. Also preferred are aromatic compounds with at least one free hydroxyl-group, especially preferred at least two free hydroxy groups that are directly aromatically coupled, wherein it is especially preferred if at least two free hydroxy-groups are coupled directly to an aromatic ring, and more especially preferred, positioned relative to each other in meta position. It is preferred that the aromatic alcohols are selected from phenols, cresoles (o-, m-, and p-cresol), naphthols (α and β-naphthol) and thymol, as well as ethylphenols, propylphenols, fluorphenols and methoxyphenols.

In accordance with the present invention preferred aromatic alcohols are those that are utilized for the production of polycarbonate-plastic material (Le. for Compact Discs, plastic bowls, baby bottles), and epoxy resin lacquers (for example, for coatings of tin cans and foil packaging), preferably 2,2-bis-(4-hydroxyphenyl)-propane (bisphenol A)

Especially preferred is the selection of the presently discussed aromatic alcohol according to the present invention from phenols with two or more hydroxy groups, preferably from brenzcatechin (pyrocatechol), resorcinol, hydroquinone and 1,4 naphthohydroquinone, phloroglucinol, pyrrogallol, hydroxyhydroquinone wherein resorcinol and/or phloroglucinol are especially preferred as aromatic alcohols.

In one embodiment, the microcapsules according to the present invention result from the use of the aromatic alcohol such as ether, wherein the ether, in a preferred embodiment, is a derivative of each of the free forms of the aromatic alcohol to be reacted according to the invention. The free alcohol can also be present, so that a mixture will thus be provided. In that case, the molar ratio between the free form of the aromatic alcohol to be reacted according to the present invention and the listed additional component (ether form of an aromatic alcohol) is preferably between 0:100, preferred 1:1, or 1:2 or 1:4.

The advantage of the mixture of aromatic alcohol with an ether form is the influence it has on the reactivity of the system, in particular, through suitable selection of conditions, a system can be created whose reactivity is in a balanced relationship to the storage stability of the system.

Esters are preferred as derivatives of aromatic alcohols.

According to the present invention, aliphatic as well as aromatic aldehydes with at least 2 C-atoms are preferred.

Especially preferred are aldehydes selected from one or more of the following groups, valeraldehyde, capronaldehyde, caprylaldehyde, decanal, succindialdehyde, cyclohexanecarbaldehyde, cyclopentanecarbaldehyde, 2-methyl-1-propanal, 2-methylpropioaldehyde, acetaldehyde, acrolein, aldosterone, antimycin A, 8′-apo-β-carotene-8′-al, benzaldhyde, butanal, chloral, citral, citronellal, crotonaldehyde, dimethylaminobenzaldehyde, folic acid, fosmidomycin, furfural, glutardialdehyde, glyceraldehyde, glycoaldehyde, glycoxal, glycoxilic acid, heptanal, 2-hydroxybenzaldehyde, 3-hydroxybutanal, hydroxymethylfurfural, 4-hydorxynonenal, isobutanal, isobutyraldehyde, methacrolein, 2-methylundecanal, nnucochloric acid, N-methylformamide, 2-nitrobenzaldehyde, nonanal, octanal, oleocanthal, orlistat, pentanal, phenylethanal, phycocyanine, piperonal, propanal, propenal, protocatechualdehyde, retinal, salicylaldehyde, secologanin, streptomycin, strophanthidin, tylosin, vanillin, cinnamic aldehyde.

Within the scope of the present invention, the aldehyde components can exhibit at least one or two, especially preferred two, three or four, more especially preferred two free aldehyde groups per molecule, wherein it is especially preferred that the provided aldehyde component is at least glycoxal, glutar-and/or succindialdehyde, especially preferred glutardialdehyde.

The molar ratio in the microcapsules according to the present invention of a) the at least one aromatic alcohol or (ether or derivative therefrom), to b) the at least one aldehyde component, can generally be between 1:1 and 1:5 especially preferred between 1:2 and 1:3 and more especially preferred with resorcinol, at about 1:2.6. The weight ratio of the components a)+b) to c) that is, the ratio of the sum of the weight of a)+b) to the weight of the component c) is generally between 1:1 and 1:0.01 especially preferred between 1:0.2 and 1:0.05.

The optionally used (meth)acrylate-polymers can be homo-or copolymers of methacrylate-monomers and/or acrylate-monomers. The term “(meth)acrylate” in this application means methacrylate as well as acrylate. The (meth)acrylate-polymers are for example homo-or copolymers, preferred copolymers of one or more polar functionalized (meth)acrylate-monomers, such as sulfonic acid groups-containing, carbonic acid groups-containing, phosphoric acid groups-containing nitril groups-containing, phosphoric acid groups-containing, ammonia groups-containing, amino groups-containing or nitrate groups-containing (meth)acrylate-monomers. In this context, the polar groups can also be present in the form of salts. The (meth)acrylate-monomers are suitable as protective colloids and can be advantageously utilized in the production of microcapsules.

(Meth)acrylate-copolymers, for example, can be composed from one or more (meth)acrylate monomers (e.g. acrylate+2-acrylamido-2-methyl-propanesulfonic acid) or from one or more (meth)acrylate-monomers and one or more different (meth)acrylate-monomers for example (methacrylate+stryrene).

Examples for (meth)acrylate-polymers are homopolymers of sulfonic acid groups containing (meth)acrylates (for example, 2-acrylamido-2 methyl-propanesulfonic acid or its salts (AMPS), commercially available as Lupasol®PA 140, BASF) or their copolymers, copolymers from acrylamide and (meth)acrylic acid, copolymers of alkyl-(meth)acrylates and N-vinylpyrrolidon (commercially available as Luviskol®K15 K30 or K90, BASF), copolymers of (meth)acrylates with polycarboxylate or polystyrenesulfonate, copolymers of (meth)acrylate with vinylethers and/or maleinic acid anhydride, copolymers of (meth)acrylates with ethylene and/or maleinic acid anhydride, copolymers of (meth)acrylates with isobutylene and/or maleinic acid anhydride or copolymers of (meth)acryclate with styrene-maleinic acid anhydride.

Preferred (meth)acrylate-polymers are homo-or copolymers, preferred copolymers of 2-acrylamido-2-methylpropanesulfonic acid of their salts (AMPS). Preferred are copolymers of 2-acrylamido-2-methyl-propanesulfonic acid or their salts. For example, copolymers with one or more comonomers from the group of (meth)acrylate of vinyl compounds such as vinylester or styrene, of the unsaturated di-or polycarbonic acid, such as maleinic acid ester or the salts of amyl compounds or allyl compounds. Certain AMPS-copolymers are novel and are also subject of the present invention. Listed in the following paragraphs are preferred comonomers for AMPS, these comonomers could be however also copolymerized with other polar functionalized (meth)acrylate-monomers.

1) Vinyl compounds, for example vinylester such as vinylacetate, vinyllaurate, vinylpropionate or vinylester or neononanic acid or aromatic vinyl compounds such as styrene comonomer, for example, styrene, alpha-methylstyrene or polar functionalized styrene such as styrene with hydroxyl, amino, nitril-, carbonic-, phosphonic acid-, phosphoric acid, nitro-or sulfonic-acid groups and their salts, wherein styrene is preferably polar functionalized in para-position.

2) Unsaturated di-or polycarbonic acids, for example maleinic acid ester such as dibutylmaleinate or dioctyirnaleinate as salts of allyl compounds, for example sodium sulfonate as salt of amyl derivatives i.e. sodium amylsulfonate.

3) (Meth)acrylate-comonomers, these are esters of acrylic acid and methacrylic acid, wherein the ester groups, for example, are saturated or unsaturated, straight chain or branched or cyclic hydrocarbon residues, which contain one or more heteroatoms such as N, O, S, P, F, CI, Br, I. Examples of such hydrocarbon residues are straight chained, branched or cyclic alkyl, straight chain, branched or cyclic alkenyl, aryl, such as phenyl or heterocylyl such as tetrahydrofurfuryl.

The (meth)acrylate-comonomer, preferred as AMPS are as follows:

a) Acrylic acid, C₁-C₁₄-alkyl-acrylic acid such as methacrylic acid,

b) (Meth)acrylamide such as acrylamide, methacrylamide, diacetone-acrylamide, diacetone-methacrylamide, N-butoxymethyl-acrylamide, N-isobutoxymethyl-acryalamide, N-butoxymethyl-methacryalamide, N-isobutoxymethyl-methacrylamide, N-methylol-acrylamide, N-methylol-methacrylamide;

c) Heterocyclyl-(meth)acrylate such as tetrahydrofurfuryl-acrylate and tetrahydrofurfurylmethacrylate or carbocyclic (meth)acrylate such as isobornyl-acrylate and isobornyl-methacrylate,

d) Urethane (meth)acrylate such as diurethanacrylate and diurethanemethylacrylate (CAS:72869-86-4).

e) C₁-C₁₄ alkylacrylate such as methyl-, ethyl, n-propyl-, n-butyl-, sec. butyl-iso-butyl-, tert. butyl-, n-pentyl-, iso-pentyl-, hexyl- (for example n-hexyl, iso-hexyl or cyclohexyl) heptyl-, octyl-, (for example, 2-ethylhexyl), nonyl-, decyl- (for example, 2-propylheptyl or iso-decyl), undecyl-, dodecyl-, tridecyl-, (for example iso-tridecyl), and tetradecyl-acrylate; the alkyl groups can be substituted optionally with one or more halogen atoms (for example fluorine, chlorine, bromine or iodine), for example tri-fluoroethyl-acrylate or with one or more amino groups, for example diethylaminoethyl-acrylate, or with one or more alkoxy groups such as methoxypropyl-acrylate or with one or more aryloxy groups such as phenoxyethyl-acrylate.

f) C₂-C₁₄ alkenylacrylate such as ethenyl-, p-propenyl-, isopropenyl-, n-butenyl-, sec. butenyl-, iso-butenyl-, tert. butenyl-, n-pentenyl-, iso-pentenyl-, hexenyl,- (for example, n-hexenyl, isohexenyl or cyclohexenyl) heptenyl-, octenyl, (for example 2-ethyl-hexenyl) nonenyl-, decenyl-, (for example, 2-propenyiheptyl or iso-decenyl), undecenyl-, dodecenyl-, tridecenyl-, (for example, isotridecenyl), and tetradecenyl-acrylate, and their epoxides such as glycidyl-acrylate or aziridine such as aziridine-acrylate.

g) C₁-C₁₄hydroxyalkylacrylate such as hydroxymethyl-, hydroxyethyl-, hydroxy-n-propyl-, hydroxy-iso-propyl-, hydroxy-n-nbutyl-, hydroxy-sec.butyl-, hydroxy-isobutyl-, hydroxy-tert.butyl-, hydroxy-n-pentyl-, hydroxy-iso-pentyl-, hydroxyhexyl-, (for example, hydroxy-n-hexyl, hydroxy-iso-hexyl, or hydroxy-cyclohexyl), hydroxyheptyl-, hydroxyoctyl-, (for example, 2-ethylhexyl), hydroxynonyl-, hydroxydecyl-, (for example, hydroxy-2-propylheptyl or hydroxy-iso-decyl), hydroxyundecyl-, hydroxydodecyl-, hydroxytridecyl-, (for example, hydroxy-iso-tridecyl), and hydroxytetradecyl-acrylate, wherein the hydroxy-group is preferably positioned in the end-position of the acrylate (ω-position) (for example 4-hydroxy-n-butylacrylate), or is positioned in (ω-1) position (for example) 2-hydroxy-n-propylacrylate);

h) Alkylene glycol acrylate, which contain one or more alkenyl glycol-units. Examples are i) monoalkylene glycoacrylate, such as acrylates of ethylene glycol, propylene glycol (for example 1,2- or 1 ,3-propandiol) butylene glycol (for example 1,2-, 1,3- or 1,4- butandiol, pentylene glycol (for example, 1,5 pentadiol) or hexylene glycol (for example 1,6 hexandiol) wherein the second hydroxy group is etherified or esterified, for example, by sulfuric acid, phosphoric acid, acrylic acid or methacrylic acid or ii) polyalkylene glycol acrylate such as polyethylene glycol acrylate, polypropylene glycol acrylate, whose second hydroxy group is optionally etherified or esterified, i.e. by sulfuric acid, phosphoric acid, acrylic acid or methacrylic acid.

Examples of (poly)alkenyl glycol-units with etherified hydroxygroups are C₁-C₁₄-alkyloxy-(poly)alkylene glycols (for example, C₁-C₁₄-alkyloxy-(poly)alkylene glycol acrylate, examples of (poly)alkylene glycol units with esterified hydroxy groups are sulfonium-(poly)alkylene glycols (for example, sulfonium-(poly)alkylene glycol acrylate and their salts, (poly)alkylene glycol diacrylate such as 1,4-butanedioldiacrylate or 1,6-hexanedioldiacrylate or (poly)alkylene glycol methacrylatacrylate such as 1,4-butanediolmethacrylatacrylate or 1,6-hexandiolmethacrylatacrylate;

The polyalkylene glycol acrylates can carry an acrylate group (for example, polyethylene glycol monoacrylate, polypropylene glycol monoacrylate, polybutylene glycol monoacrylate, polypentylene glycol monoacrylate or polyhexylene glycol monoacrylate) or two or more, preferably two, acrylate groups carry such as polyethylene glycol diacrylate, polypropylene glycol diacrylate, polybutylene glycol dicarylate, polypentylene glycol diacrylate or polyhexylene glycol diacrylate;

The polyalkylene glycol acrylate can also contain two or more polyalkylene glycol blocks, for example, blocks of polymethylene glycol and polyethylene glycol or blocks of polyethylene glycol and polypropylene glycol;

The degree or polymerization of the poly alkylene glycol-units or poly alkylene-blocks are generally in the range from 1 to 20, preferably in the range from 3 to 10, especially preferred in the range from 3 to 6.

C₁-C₁₄-alkylmethacrylate such as methyl-, ethyl-,n-propyl-, iso-propyl-, n-butyl-, sec. butyl-, iso-butyl-, tert. butyl-, n-pentyl-, iso-pentyl, hexyl- (for example n-hexyl, iso-hexyl or cyclohexyl), heptyl-, octyl-, (for example, 2-ethylhexyl), nonyl-, decyl- (for example, 2-propylheptyl or iso-decyl), undecyl-, dedecyl-, tridecyl-, (for example, iso-tridecyl), and tetradecylmethacrylate; the alkyl groups can be optionally substituted with one or more halogen atoms (for example, fluorine, chlorine, bromine or iodine), i.e. trifluoroethyl-methacrylate or with one or more amino groups, for example diethylaminoethylmethacrylate or with one or more aryloxy groups such as phenoxyethylmethacrylate.

C₂-C₁₄-alkenylmethacrylate such as ethenyl-, n-propenyl-, iso-propenyl, n-butenyl-, sec. butenyl, iso-butenyl-, tert. butenyl-, n-pentenyl-, iso-pentenyl-, hexenyl- (for example, n-hexenyl, iso-hexenyl or cyclohexenyl), heptenyl-, octenyl-, (for example, 2-ethylhexenyl), nonenyl-, decenyl- (for example, 2-propenylheptyl or iso-decenyl) undecenyl-, dodecenyl-, tridecenyl-, (for example iso-tridecenyl), and tetradecenyl-methacrylate and their epoxies such as glycidyl-methacrylate or aziridine such as aziridine-methacrylate.

C₁-C₁₄-hydroxyalkylmethacrylate such as hydroxymethyl-. hydroxyethyl-, hydroxy-n-propyl-, hydroxy-iso-propyl-, hydroxy-n-butyl-, hydroxy-sec.butyl-, hydroxy-iso-butyl-, hydroxy-tert.-butyl-, hydroxy-n-pentyl-, hydroxy-iso-pentyl-, hydroxyhexyl- (for example, hydroxy-n-hexyl, hydroxy-iso-hexyl or hydroxy-cyclo-hexyl), hydroxy-heptyl-, hydroxy-octyl-, (for example, 2-ethylhexyl), hydroxynonyl,-, hydroxydecyl-, (for example, hydroxyl-2-propylheptyl or hydroxyl-iso-decyl), hydroxyundecyl-, hydroxydodecyl-, hydroxytridecyl- (for example, hydroxy-iso-tridecyl), and hydroxytetradecyl-methylacrylate, wherein the hydroxyl group is preferably in the end-position ((ω-position) (for example, 4-hydroxy-n-butylrnehtacrylate) or in (ω-1) position (for example, 2-hydroxy-n-propylmethacrylate of the alkyl residue;

Alkylene glycol methacrylate which contain one or more alkylene-units. Examples are i) monoalkylene glycol methacrylate, such as methylacrylate of ethyl glycol, propylene glycol (for example, 1,2- or 1,3-propandiol), butylene glycol (for example, 1,2-, 1,3-, or 1,4-butandiol, pentylene glycol (for example, 1,5 pentadiol) or hexyleneglycol (for example, 1,6 hexanediol), where the second hydroxyl-group is etherified or esterified, for example with sulfonic acid, phosphoric acid, acrylic acid or methacrylic acid, or ii) polyalkylene glycol methacrylate such as polyethylene glycol methacrylate, polypropylene glycol methacrylate, polybutylene glycol methacrylate, polypentylene glycol methacrylate, polypropylene glycol methacrylate, polybutylene glycol methacrylate, polypentylene glycol methacrylate or polyhexylene glycol methacrylate, whose second hydroxy group is optionally etherified or esterified, for example, with sulfonic acid, phosphoric acid, acrylic acid or methacrylic acid;

Examples of (poly)alkylene glycol-units with etherified hydroxy groups are C₁-C₁₄-alkoxy(poly) alkylene glycols (for example, C₁-C₁₄- alkyl-(poly)alkylene glycol methacrylate), examples of (poly)alkylene glycol-units with esterified hydroxy groups are sulfonium-(poly)alkylene glycols (for example, sulfonium-(poly)alkylene glycol methacrylate) and their salts or (poly)alkylene glycol dimethylacrylate such as 1,4-butanedioldimethacrylate.

The polyalkylene glycol methacrylates can carry a methacrylate group (for example, polyethylene glycol monomethacrylate, polypropylene glycol mono methacrylate, polybutylene glycol mono-methacrylate, polypentylene glycol mono-methacrylate or polyhexylene glycol monomethacrylate) two or more, preferably two, methacrylate groups carry, such as polyethylene glycol dimethylacrylate, polypropylene glycol dimethacrylate, polybutylene glycol dimethacrylate, polypentylene glycol dimethacrylate or polyhexylene glycol dimethacrylate;

The polyalkylene glycol methacrylates can also include two or more different polyalkylene glycol blocks, for example, blocks of polymethylene glycol and polyethylene glycol or blocks of polyethylene glycol and polypropylene glycol (for example, bisomer PEM63PHD (Cognis), CAS 58916-75-9);

The degree of polymerization of the polyalkylene glycol-units or polyalkylene glycol blocks are generally within the range from 1 to 20, preferably in the range from 3 to 10, especially preferred in the range from 3 to 6.

Examples of preferred (meth)acrylate-comonomers are listed as follows.

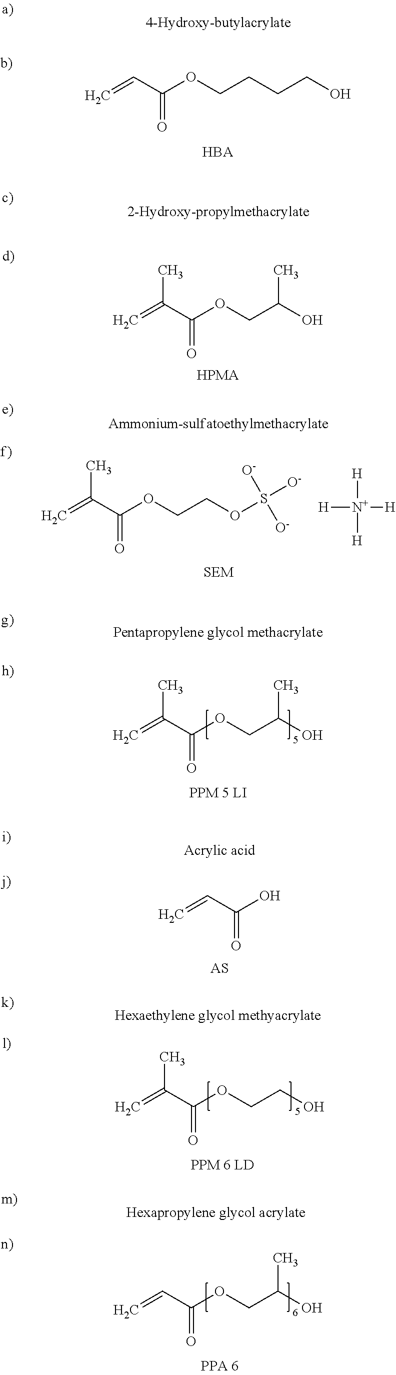

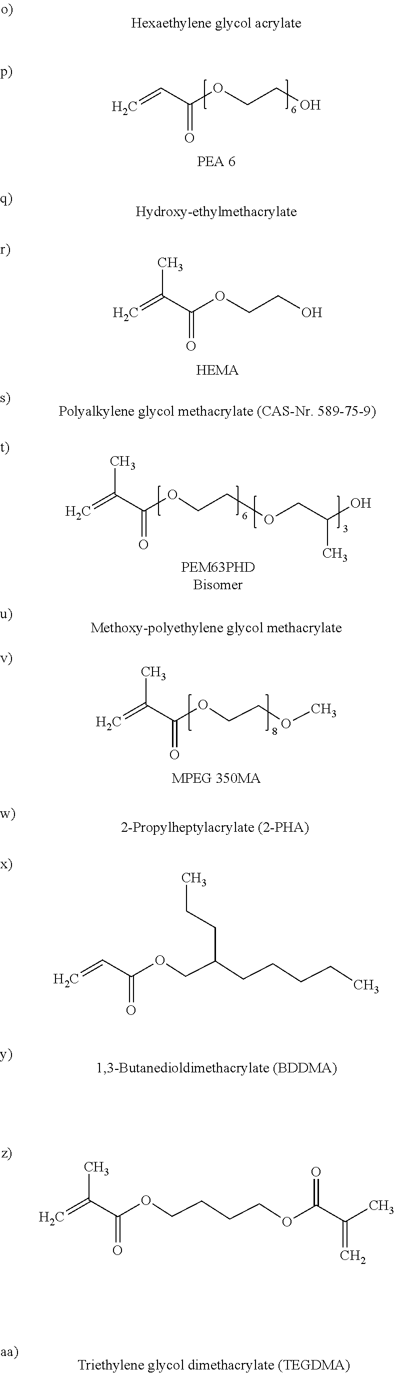

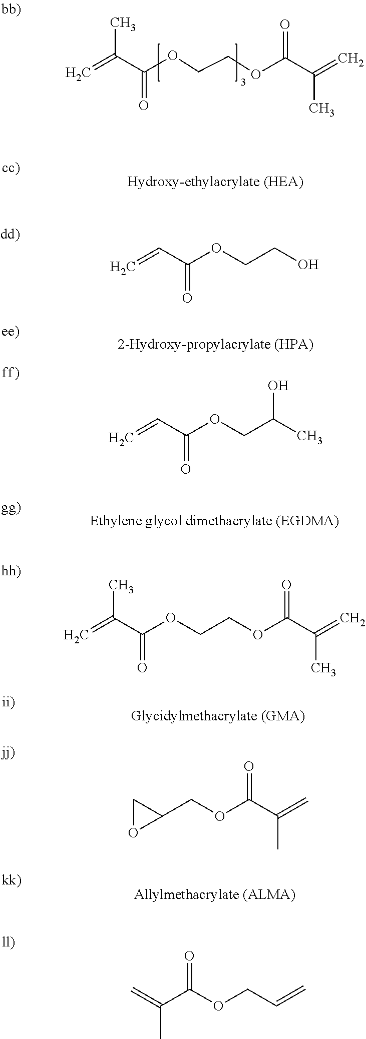

The AMPS-copolymers generally exhibit a portion of AMPS-units of greater than 50-Mol %, preferably in the range from 60-95 Mol-%, especially preferred from 80 to 99 Mol-%, the portion of comonomers is generally smaller than 50 Mol-%, preferably in the range from 5 to 40 Mol-%, especially preferred from 1 to 20 Mol %.

The copolymers can be obtained by known methods, for example by a batch-or semibatch-method. For example, suitable amounts of water and monomers are first fed to a temperature controllable reactor and placed under an inert gas atmosphere. The feed is then stirred and brought to reaction temperature (preferably in the range of about 70-80° C.) and then initiator added, preferably in an aqueous solution. Suitable initiators are known for radicalic polymerizations, for example, sodium-, potassium- or ammonium peroxodisulfate, or H₂O₂ mixtures, for example mixtures of H₂O₂ with citric acid. After the maximal temperature has been reached and as soon as it is lowering either a) the remaining monomers are added with the after-reaction following (semibatch method) or b) the after-reaction follows directly (batch method). Subsequently, the resulting reaction mixture is cooled to room temperature and the copolymer isolated from the aqueous solution, for example, by extraction with organic solvents, such as hexane or methylene chloride, with subsequent removal of the solvent by distillation. Thereafter, the copolymer is washed with organic solvents and dried. The resulting reaction mixture can be further treated, in which case it is advantageous to add a preservative to the aqueous copolymer solution,

The AMPS-copolymers are suitable as protective colloids in the production of microcapsules. Various of the AMPS-copolymers described are novel and are subject of the present invention, as well as the use of these copolymers for the production of microcapsules, for example microcapsules from phenol-aldehyde-polymers such as phenol-formaldhyde-polymers, melamine-formaldehyde-polymers, polyurethanes, gelatins, polyamides or polyureas. Preferably the copolymers according to the present invention are suitable as protective colloids for the production of microcapsules of the present invention.

Preferred microcapsules of the present invention comprise the following components a) b) and c):

Phloroglucinol, glutardialdehyde, AMPS/hydroxyethylmethacrylate-copolymer;

Phloroglucinol, succindialdehyde, AMPS/hydroxyethylmethacrylate-copolymer;

Phloroglucinol, glyoxal, AMPS/hydroxyethylmethacrylate-copolymer;

Phloroglucinol, glutardialdehyde, AMPS/hydroxyethylacrylate-copolymer;

Phloroglucinol, succindialdehyde, AMPS/hydroxyethylacrylate-copolymer;

Phloroglucinol, glyoxal, AMPS/hydroxyethylacrylate-copolymer;

Phloroglucinol, glutardialdehyde, AMPS/hydroxypropylmethacrylate-copolymer;

Phloroglucinol, succindialdehyde, AMPS/hydroxypropylmethacrylate-copolymer;

Phloroglucinol, glyoxal, AMPS/hydroxypropylmethacrylate-copolymer;

Phloroglucinol, glutardialdehyde, AMPS/hydroxypropylacrylate-copolymer;

Phloroglucinol, succindialdehyde, AMPS/hydroxypropylacrylate-copolymer;

Phloroglucinol, glyoxal, AMPS/hydroxypropylacrylate-copolymer;

Phloroglucinol, glutardialdehyde, AMPS/hydroxybuylmethacrylate-copolymer;

Phloroglucinol, succindialdehyde, AMPS/hydroxybutylmethacrylate-copolymer;

Phloroglucinol, glyoxal, AMPS/hydroxybutylmethacrylate-copolymer;

Phloroglucinol, glutardialdehyde, AMPS/hydroxybutylacrylate-copolymer;

Phloroglucinol, succindialdehyde, AMPS/hydroxybutylacrylate-copolymer;

Phloroglucinol, glyoxal, AMPS/hydroxybutylacrylate-copolymer;

Phloroglucinol, glutardialdehyde, AMPS/polyethylene glycol monomethacrylate-copolymer;

Phloroglucinol, succindialdehyde, AMPS/polyethylene glycol monomethacrylate-copolymer;

Phloroglucinol, glyoxal, AMPS/polyethylene glycol monomethacrylate-copolymer;

Phloroglucinol, glutardialdehyde, AMPS/polyethylene glycol monoacrylate-copolymer;

Phloroglucinol, succindialdehyde, AMPS/polyethylene glycol monoacrylate-copolymer;

Phloroglucinol, glyoxal, AMPS/polyethylene glycol monoacrylate-copolymer;

Phloroglucinol, glutardialdehyde, AMPS/polypropylene glycol monomethacrylate-copolymer;

Phloroglucinol, succindialdehyde, AMPS/polypropylene glycol monomethacrylate-copolymer;

Phloroglucinol, glyoxal, AMPS/polypropylene glycol monomethacrylate-copolymer;

Phloroglucinol, glutardialdehyde, AMPS/polypropylene glycol monoacrylate-copolymer;

Phloroglucinol, succindialdehyde, AMPS/polypropylene glycol monoacrylate-copolymer;

Phloroglucinol, glyoxal, AMPS/polypropylylene glycol monoacrylate-copolymer;

Phloroglucinol, glutardialdehyde, AMPS/methoxy-polyethylene glycol monomethacrylate-copolymer;

Phloroglucinol, succindialdehyde, AMPS/methoxy-polyethylene glycol monomethacrylate-copolymer;

Phloroglucinol, glyoxal, AMPS/methoxy-polyethylene glycol monomethacrylate-copolymer;

Phloroglucinol, glutardialdehyde, AMPS/methoxy-polyethylene glycol monoacrylate-copolymer;

Phloroglucinol, succindialdehyde, AMPS/ methoxy-polyethylene glycol monoacrylate-copolymer;

Phloroglucinol, glyoxal, AMPS/ methoxy-polyethylene glycol monoacrylate-copolymer;

Resorcinolol, glutardialdehyde, AMPS/hydroxyethylmethacrylate-copolymer;

Resorcinol, succindialdehyde, AMPS/hydroxyethylmethacrylate-copolymer;

Resorcinol, glyoxal, AMPS/hydroxyethylmethacrylate-copolymer;

Resorcinol, glutardialdehyde, AMPS/hydroxyethylacrylate-copolymer;

Resorcinol, succindialdehyde, AMPS/hydroxyethylacrylate-copolymer;

Resorcinol, glyoxal, AMPS/hydroxyethylacrylate-copolymer;

Resorcinol, glutardialdehyde, AMPS/hydroxypropylmethacrylate-copolymer;

Resorcinol, succindialdehyde, AMPS/hydroxypropylmethacrylate-copolymer;

Resorcinol, glyoxal, AMPS/hydroxypropylmethacrylate-copolymer;

Resorcinol, glutardialdehyde, AMPS/hydroxypropylacrylate-copolymer;

Resorcinol, succindialdehyde, AMPS/hydroxypropylacrylate-copolymer;

Resorcinol, glyoxal, AMPS/hydroxypropylacrylate-copolymer;

Resorcinol, glutardialdehyde, AMPS/hydroxybutylmethacrylate-copolymer;

Resorcinol, succindialdehyde, AMPS/hydroxybutylmethacrylate-copolymer;

Resorcinol, glyoxal, AMPS/hydroxybutylmethacrylate-copolymer;

Resorcinol, glutardialdehyde, AMPS/hydroxybutylacrylate-copolymer;

Resorcinol, succindialdehyde, AM PS/hydroxybutylacrylate-copolymer;

Resorcinol, glyoxal, AMPS/hydroxybutylacrylate-copolymer;

Resorcinol, glutardialdehyde, AMPS/polyethylene glycol monomethacrylate-copolymer;

Resorcinol, succindialdehyde, AMPS/polyethylene glycol monomethacrylate-copolymer;

Resorcinol, glyoxal, AMPS/polyethylene glycol monomethacrylate-copolymer;

Resorcinol, glutardialdehyde, AMPS/polyethylene glycol monoacrylate-copolymer;

Resorcinol, succindialdehyde, AMPS/polyethylene glycol monoacrylate-copolymer;

Resorcinol, glyoxal, AMPS/polyethylene glycol monoacrylate-copolymer;

Resorcinol, glutardialdehyde, AMPS/polypropylene glycol monomethacrylate-copolymer;

Resorcinol, succindialdehyde, AMPS/polypropylene glycol monomethacrylate-copolymer;

Resorcinol, glyoxal, AMPS/polypropylene glycol monomethacrylate-copolymer;

Resorcinol, glutardialdehyde, AMPS/polypropylene glycol monoacrylate-copolymer;

Resorcinol, succindialdehyde, AMPS/polypropylene glycol monoacrylate-copolymer;

Resorcinol, glyoxal, AMPS/polypropylene glycol monoacrylate-copolymer;

Resorcinol, glutardialdehyde, AMPS/methoxy-polyethylene glycol monomethacrylate-copolymer;

Resorcinol, succindialdehyde, AMPS/methoxy-polyethylene glycol monomethacrylate-copolymer;

Resorcinol, glyoxal, AMPS/methoxy-polyethylene glycol monomethacrylate-copolymer;

Resorcinol, glutardialdehyde, AMPS/methoxy-polyethylene glycol monoacrylate-copolymer;

Resorcinol, succindialdehyde, AMPS/methoxy-polyethylene glycol monoacrylate-copolymer;

Resorcinol, glyoxal, AMPS/methoxy-polyethylene glycol monoacrylate-copolymer;

In one embodiment of the present invention, additionally one or more nitrogen-containing or silica dioxide-containing agents can be utilized for the production of the microcapsules according to the present invention. Thereby, the nitrogen-containing agents can be polymerized into the resin (for example, to enhance the characteristics of the resins) or utilized for after-treatment.

Preferably, heterocyclic compounds with at least one nitrogen atom as a heteroatom, which is either adjacent to an amino substituted carbon atom, or a carbonyl group, such as for example, pyridazin, pyrimidin, pyrazin, pyrrolidon, amino pyridine, and compounds that are derived therefrom. Principally, all amino pyridines are suitable, such as for example, melamine, 2,6-diaminopyridin, substituted and dimer amino pyridines and mixture from these compounds. Advantageous are furthermore polyamides and dicyandiamide, urea and its derivatives as well as pyrrolidon and compounds derived therefrom. Examples of suitable pyrrolidons are for example imidazolidinon and compounds derived therefrom, such as for example hydantoin, derivatives of which are especially advantageous, and especially advantageous are compounds from allantonin and its derivatives. Especially preferred are furthermore triamino-1, 3, 5-triazin (melamine) and its derivatives.

It should be especially emphasized that the after-treatment involves “purely” an after-treatment of the surface in order to realize this particularly preferred embodiment. In other words: in this preferred embodiment, the recited nitrogen-containing agent is not involved in the generation of the structure of the entire capsule walls but is predominantly concentrated on the exterior surface of the capsule walls The after-treatment can also be carried out with silica gel (preferably amorphous hydrophobic silica gel) or with aromatic alcohols a), wherein those are preferably utilized as a slurries.

A further subject of the present invention is microcapsule dispersions which contain one or more of the microcapsules according to the present invention. Subject of the present invention is also the use of the aromatic alcohol to be reacted according to the present invention (or its derivative, in particular, ether), for reacting with aldehyde components according to the present invention for the formation of capsule walls of microcapsules. Thereby, the free alcohol or its ether can be available as a mixture. It is preferred, according to the use of the present invention, that formaldehyde-free microcapsules are provided. Small amount of formaldehyde can however be added to the mixture, generally less than 0.05 Mol-weight relative to the entire reaction, for example as a preservative.

The present invention also includes a method for the production of the microcapsules according to the present invention, wherein the at least one aromatic alcohol to be reacted according to the present invention with the at least one aldehyde component to be reacted according to the present invention has at least two C-atoms per molecule and optionally at least one (meth)acrylate polymer, as appropriate, in the presence of at least one substance to be made into capsules (core substance), are reacted together—and then by later raising the temperature, realizing hardening of the capsules. It is especially preferred that during the process the pH value is elevated.

The framework of the method of the present invention preferably includes the following steps:
a) the at least one aromatic alcohol and/or its derivative or ether and the at least one aldehyde component and optionally at least one (meth)acrylate polymer and at least one substance to be made into capsules at a temperature from 40 to 60° C. and a pH-value between 6 and 9, preferably 7 and 8.5 are mixed together and
b) in a later step, at a temperature from 40 to 65° C., the pH-value raised to above 9, preferably between 9.5 and 11, wherein
c) later, hardening of the capsules is carried out by raising the temperature to 60° C. to 110° C., preferably 70° C. to 90° C., especially at 80° C.

If phloroglucinol is used as an alcohol component, then hardening is advantageously carried out with acids; the preferred pH-value is then maximally 4, especially preferred between 3 and 4, for example between 3.2-3.5.

The yield and quality of the microcapsules or microcapsule dispersions according to the present invention can be influenced by the selected parameters of temperature, pH-value and/or stirring speed. In particular, too low a temperature can lead to capsule walls that are not suitably dense. The expert can detect this because of a reduced yield as well noticing precipitation of core material as a condensate in the filter of the drier. Alternatively, it must be made sure that the reaction speed is not too high, as this causes that too little material deposited around the capsules, or that too much wall material remains free and undeposited. This free wall material can then be present as particles of a size greater than the capsules themselves.

The alkalinity can also be important for the quality of the microcapsules according to the present invention. Besides that, within the framework of carrying out the process, the pH-value causes a tendency of the batch to gelatinize. If the particle formation (step b) above) is carried out at a pH-value of 9 or less, the batch could gelatinize.

In one embodiment of the method according to the present invention, an alkaline salt, preferably alkali carbonate is used in order to control the alkalinity, especially sodium carbonate. Sodium carbonate is preferred as it reduces the possibility to gelatinize.

It is within the scope of the method of the present invention that, at the start of the reaction (process step a) the aromatic alcohol is stirred together with the aldehyde component, wherein the stirring speed is at 500 to 2500 rpm, especially at 1000 to 2000 rpm. To the resulting pre-condensate, subsequently, the (meth)acrylate-polymer is optionally added to the substance to be capsulated. Preferably, later, directly before or while the alkalinity (method step b) is being raised the stirring speed is increased to 3000 to 5000 rpm, especially to 3500 to 4500 rpm, predominantly at 4000 rpm.

Preferably, the increased stirring speed is maintained until the viscosity values of the mixture decrease, wherein after the viscosity starts to drop, the stirring speed is lowered, preferably to 500 to 2500 rpm, especially preferred to 1000 to 2000 rpm. Any sooner decrease of the stirring speed can also lead to undesirable gelatinizing of the batch.

Preferably, after the start of the afore-described reduction of the viscosity, at least 20 minutes, especially preferred between 30 and 180 minutes, at a stirring speed of 1000 to 2000 rpm and a temperature of 40 to 65° C., stirring continues, before method step c) hardening of the capsules is carried out by raising the temperature. This phase, after the start of the afore-described reduction in viscosity and before hardening of the capsules, is also designated as the resting phase. The resting phase can preferably serve to realize the pre-formation of suitably stable capsule walls, in other words, to form the capsule walls in a stable manner so that no core material is able to escape.

A further subject of the present invention is the use of the microcapsules or microcapsules dispersions according to the present invention for the controlled release of core material, preferably selected from such agents as aromatics, pesticides, herbicides, greasing agents, lubricants, insecticides, antimicrobial agents, pharmaceutical agents, cosmetic agents, latent heat storing agents (for example waxes), catalysts, (for example organic carbonates), self-healing agents (for example norbornes, dicyclopentadiene) coating systems such as lacquers (for example, aromatics, lacquers), hydrophobic waxes, hydrophobic ene-components or hydrophobic solvents.

In addition, subject of the present invention are products that comprise microcapsules or microcapsule dispersions according to the present invention, the use of which is preferably in fields of application selected from the areas of lacquer technology, construction chemistry, dental technology, preferably as a component for fast hardening tooth filling material, self-healing systems, cosmetics, preferably for scented and aromatic oils, pharmaceutical, preferably as a carrier, medical technology, laundering, cleaning disinfecting, gluing, treatment of plants, preferably fungicides, pesticides, insecticides, herbicides or corrosion protection.

Generally, the microcapsules have an average diameter of 1-1000 μm. The term microcapsules as used herein also include nano capsules, that is, capsules having an average diameter <1 μm. The capsules preferably have an average diameter of 0.1 to 100 μm. The wall thickness can be for example 0.05 to 10 μm.

The production of solid spheres is also possible, that is, capsules which do not surround core material. These solid spheres can even have an average diameter of less 500 nm (preferably between 300 and 400 nm). Preferably, these can be mono dispersed solid spheres. For the production of an embodiment of these spheres phloroglucinol can be used.

The solid spheres according to the present invention can have application as standard or control batches, for example, in the medical technology field (for example, as a calibration device in particle sizers or erythrocyte counters) or as an abrasive component in scrubbing agents, for decorative effects or as a distance holder for printing lacquers with pressure sensitive particles.

### EXAMPLE 1

### Production of Copolymers

a) AMPS-Hydroxybutylacrylate for the 1500 g batch, 891 g demineralized water combined with 585 g AMPS (50% aqueous solution) and 7.5 g 4-hydroxybutylacrylate (HBA) is filled into the reactor and placed under protective gas atmosphere. The reaction mixture is heated under stirring (400 rpm) to 75° C.). 0.03 g of the water soluble initiator sodium peroxodisulfate is dissolved in 15 g of water and injected into the reactor by means of an injection needle when the reaction temperature has been reached. After reaching the maximal temperature, an hour long after-reaction is started. Subsequently, the batch is cooled at room temperature and 1.5 g of preservative added.

The aqueous solution is then characterized by viscosity, solid content and pH-value. The viscosity is 540 mPas (measured by 20 rpm Brookfield), the solid content is 21% and the pH-value is at 3.3. Then, 3 g are placed into a Petri dish and dried for 24 hours at 160° C. in the drying chamber. The end weight is 0.69 g corresponding to a yield of 21.6%.

b) AMPS-Polyalkylene Glycolmonomethacrylate.

The feed comprises 912 g de-mineralized water, 240 g AMPS and 7.5 g poly(ethylene/propylene) glycolmonomethacrylate (Bisomer PEM HD from Cognis CAS-No.: 589-75-9). The mixture is placed under protective gas atmosphere. The reaction mixture is heated under stirring (400 rpm) to 75° C. 1.5 g of sodium peroxodisulfate are dissolved in 15 g water and injected into the reactor by means of an injection needle. After the temperature in the reactor has reached its maximum and is starting to decrease, 240 g AMPS with 83 g PEM 63P HD are dosed by means of a hose pump for a period of an hour. Following, is a half hour after-reaction. Subsequently, the batch is cooled to room temperature and 1.5 g preservative added.

The aqueous solution is then characterized by viscosity, solid content and pH-value. The viscosity is 110mPas (measured by 20 rpm Brookfield), the solid content is 23% and the pH-value is at 3.1. Then 3 g are placed into a Petri dish and dried for 24 hours at 160° C. in the drying chamber. The end weight is 0.68 g corresponding to a yield of 21.6%.

### EXAMPLE 2

### Resorcinol Capsule

In a 400 ml beaker, 5.5 g resorcinol are dissolved in 70 g water under stirring (stirring speed about 1500 rpm) and thereafter 2.0 g sodium carbonate solution added (20 weight %), resulting in a pH-value at about 7.9. This solution is warmed to a temperature of about 52° C. Then, 25.5 g glutardialdehyde is added.

The mixture is stirred for about another 10 minutes at a stirring speed of about 1500 rpm and at a temperature of about 52° C. (pre-condensation). Thereafter, about 20 g water are added and about 2 minutes later 1 g of one of a protective colloid a) copolymer 1 a, b) copolymer 1b and c) poly AMPS (AMPS-homopolymer); and again about 2 minutes later 55 g palatinol A (=diethylphtalate) added. Directly following, the stirring speed is increased to about 4000 rpm and at about the same time 20.0 g of sodium carbonate solution (20% by weight) added. Afterwards, the pH-value of the mixture is about 9.7. Thereafter, the viscosity and the volume of the mixture increase. Stirring continues at a stirring speed of about 4000 rpm, until the viscosity is decreasing. Only then, the stirring speed is lowered to about 1500 rpm. At a temperature of about 52° C. and remaining stirring speed, the batch is being stirred for about another 60 minutes. This phase is the resting phase. Following, the mixture is heated to about 80° C. and the capsules hardened at this temperature across a period of 3 hours. Capsule size distribution −D (90) 5-10 μm: capsulation efficiency about 90%: Drying yield is >90%; solid body of the slurry is about 40% by weight. The choice of protective colloid and the bases and acids for the successful capsulation process spans a large range, wherein those bases are preferred that elicit catalytic effects in the reaction of the aromatic alcohols with the aldehydes. Thereby, the formation of resoles, as well as the formation of novolak analog capsule walls is realized.

The so-produced capsules are free of formaldehyde and without a problem can be further processed as stable core/shell-microcapsules from the aqueous slurry into a dry free-flowing powder.

The charging of the capsules can be realized with hydrophobic materials, gas, liquid, solid and classes of substances, which do not enter into side- or parallel reactions under suitable reaction conditions.

### EXAMPLE 3

### Production of a Solid Sphere

A solution of 4.5 g phloroglucinol, 200 g water and 32.2 g glutardialdehyde- solution (50%) is slowly stirred for 90 minutes at room temperature. Subsequently, the temperature is kept for 2 hrs at 40° C.

During this time, particles form, which in this case grow up to a size of 4 μm and which exhibit a very narrow size distribution.

These particles are subsequently hardened for 2 hours at 60° C. The finished slurry has a pH-value of 3.4.

1-40. (canceled)

41. Microcapsules having walls comprising a resin resulting from reacting:
a) at least one aromatic alcohol or its ether or derivative and
b) at least one aldehyde component having at least two C-atoms per molecule, and
c) optionally at least one (meth)acrylate-polymer selected from the group consisting of copolymers of 2-acrylamino-2-methyl-propane sulfonic acid and their salts with one or more comonmers from the group of (meth)acrylates.

42. The microcapsules according to claim 41, wherein the at least one aromatic alcohol includes per molecule at least two aromatically free hydroxy-groups.

43. The microcapsules according to claim 41, wherein the at least one aromatic alcohol is selected from the group consisting of phenols, cresols (o-, m-, and p-cresol), naphthols (α- and β-naphthols) and thymol.

44. The microcapsules according to claim 42 wherein at least two free hydroxyl-groups of the at least one aromatic alcohol are bonded directly at an aromatic ring.

45. The microcapsules according to claim 41, wherein the at least one aromatic alcohol is selected from phenols having two or more hydroxy-groups,

46. The microcapsules according to claim 41, wherein the aromatic alcohol is resorcinol and/or phloroglucinol.

47. The microcapsules according to claim 41, wherein in addition to the aromatic alcohol, the ether of the at least one aromatic alcohol is reacted as an additional component.

48. The microcapsules according to claim 41, wherein the aldehyde component is selected from aliphatic and aromatic aldehydes.

49. The microcapsules according to claim 48, wherein the aldehyde component is selected from the group consisting of valeraldehyde, capronaldehyde, caprylaldehyde, decanal, succindialdehyde, cyclohexanecarbaldehyde, cyclopentanecarbaldehyde, 2-methyl-1-propanal, 2-methylpropionaldehyde, acetaldehyde, acrolein, aldosterone, antimycin A, 8′-apo-β-carotene-8′-al, benzaldhyde, butanal, chloral, citral, citronellal, crotonaldehyde, dimethylaminobenzaldehyde, folic acid, fosmidomycin, furfural, glutaraldehyde, glyceraldehyde, glycoaldehyde, glycoxal, glycoxilic acid, heptanal, 2-hydroxybenzaldehyde, 3-hydroxybutanal, hydroxymethylfurfural, 4-hydorxynonenal, isobutanal, isobutyraldehyde, methacrolein, 2-methylundecanal, mucochioric acid, N-methylformamide, 2-nitrobenzaldehyde, nonanal, octanal, oleocanthal, orlistat, pentanal, phenylethanal, phycocyanin, piperonal, propanal, propenal, protocatechualdehyde, retinal, salicylaldehyde, secologanin, streptomycin, strophanthidin, tylosin, vanillin and cinnamic aldehyde.

50. The microcapsules according to claim 41, wherein the at least one aldehyde component includes at least two free aldehyde groups per molecule.

51. The microcapsules according to claim 41, wherein the aldehyde component includes at least two aldehyde groups.

52. The microcapsules according to claim 41, wherein at least one of glutaraldehyde and succindialdehyde is present as the aldehyde component.

53. The microcapsules according to claim 41, wherein the (meth)acrylate-polymer is a copolymer of 2-acrylamido-2-methylpropane sulfonic acid or its salts with one or more (meth)acrylate comonomers selected from the group consisting of acrylic acid, C₁-C₁₄-alkyl-acrylic acid, (meth)acrylamide, heterocyclyl-(meth)acrylate, urethane-(meth)acrylate, C₁-C₁₄-alky-(meth)acrylate, C₂-C₁₄-alkenyl-(meth)acrylate, C₁-C₁₄-hydroxyalkyl-(meth)acrylate and alkylene glycol-(meth)acrylate.

54. The microcapsules according to claim 53, wherein the (meth)acrylate-comonomers are selected from the group consisting of:

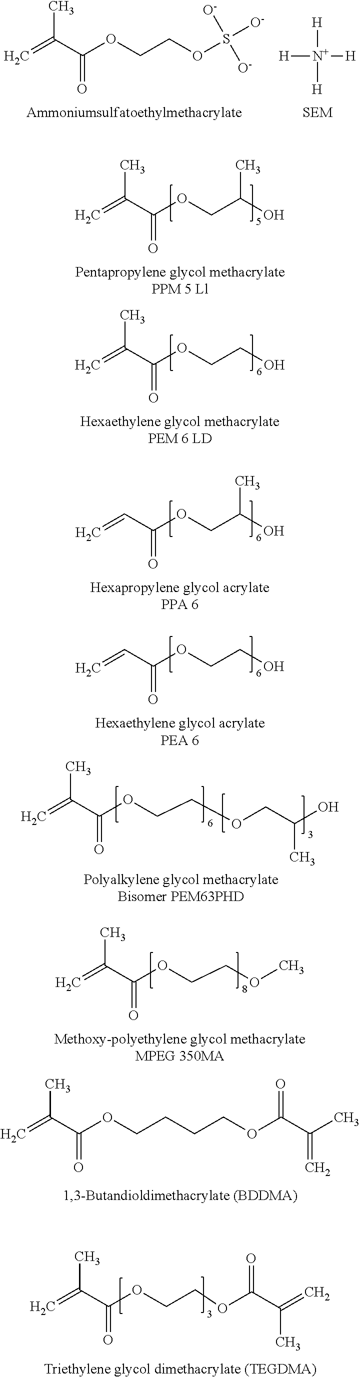

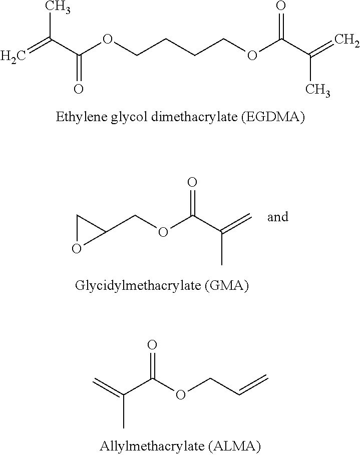

55. The microcapsules according to claim 41, wherein the molar ratio of
a) the at least one aromatic alcohol to
b) the at least one aldehyde component having at least two C-atoms per molecule,
is between 1 to 2 and 1 to 3.5.

56. The microcapsules according to claim 55, wherein the molar ratio is between 1 to 2.4 and 1 to 2.8,

57. The microcapsules according to claim 56, wherein the molar ratio is at 1 to 2.6.

58. The microcapsules according to claim 41, further comprising d) a nitrogen-containing agent.

59. The microcapsules according to claim 58, wherein the capsule surface is after-treated with melamine, with silica gel or the aromatic alcohol a).

60. A microcapsule dispersion comprising one or more microcapsules according to claim 41.

61. A copolymer comprising units derived from:
a) 2-acrylamido-2-methyl-propanesulfonic acid or its salts (AMPS), and
b) one or more (meth)acrylate-comonomers from the group of vinyl ester, urethane-(meth)acrylates, C₂-C₁₄-alkenyl-(meth)acrylates and their epoxies and arizidines, and of alkylene glycol (meth)acrylates, except copolymers comprising units of 2-acrylamido-2-methyl-propanesulfonic acid sodium and methoxy-polyethylene glycol methacrylate and of 2-acrylamido-2-methyl-propane sulfonic acid and methacrylic acid allylester.

62. The copolymer according to claim 61, wherein the alkylene glycol-(meth)-acrylate is selected from the group consisting of:

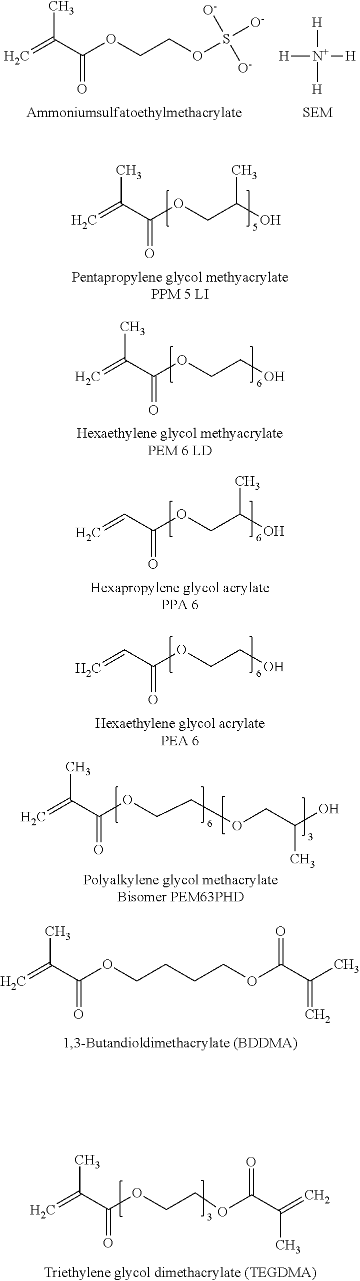

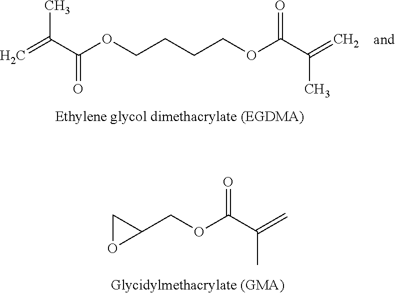

63. Use of a copolymer comprising units derived from
a) 2-acrylamido-2-methyl-propane sulfonic acid or its salts (AMPS), and
b) one or more comonomers from the group of vinylester, (meth)acrylamide, urethane-(meth)acrylate, C₂-C₁₄-alkenyl-(meth)acrylate and their epoxies and aziridines, and of alkylene glycol (meth)acrylates,
for the production of a microcapsule according to claim 41.

64. Use of the copolymer of claim 63, wherein the comonomers are selected from the group consisting of

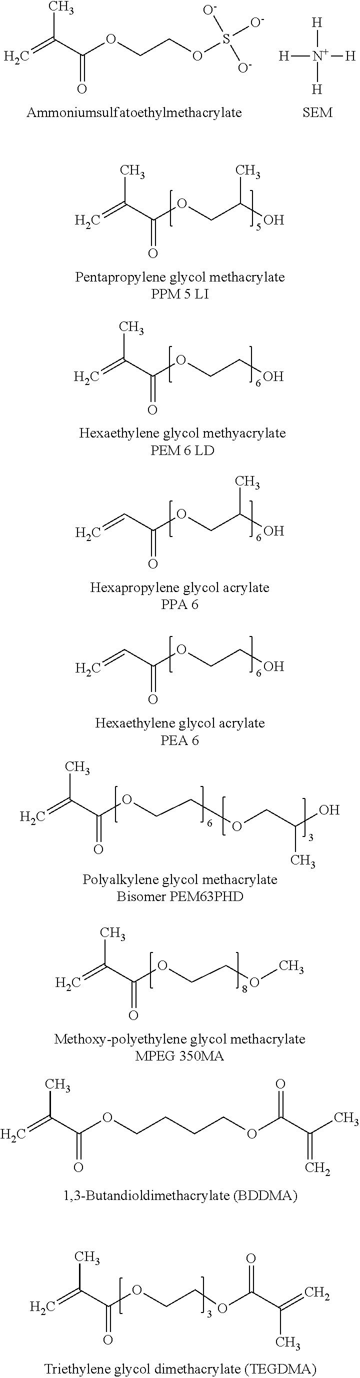

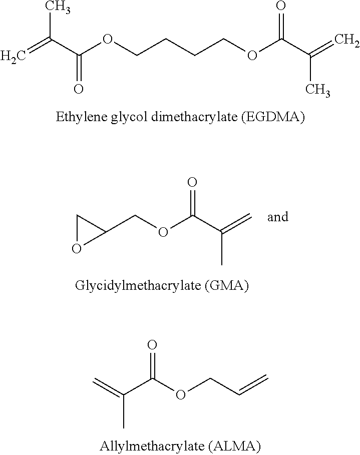

65. A method of using a copolymer comprising units derived from,
a) 2-acrylamido-2-methyl-propane sulfonic acid or its salts (AMPS), and
b) one or more comonomers from the group of vinylester, (meth)acrylamide, urethane-(meth)acrylate, C₂-C₁₄-alkenyl-(meth)acrylate and their epoxies and aziridines, and of alkylene glycol (meth)acrylates,
as protective colloid.

66. The method according to claim 65, wherein the comonomers are selected from the group consisting of,

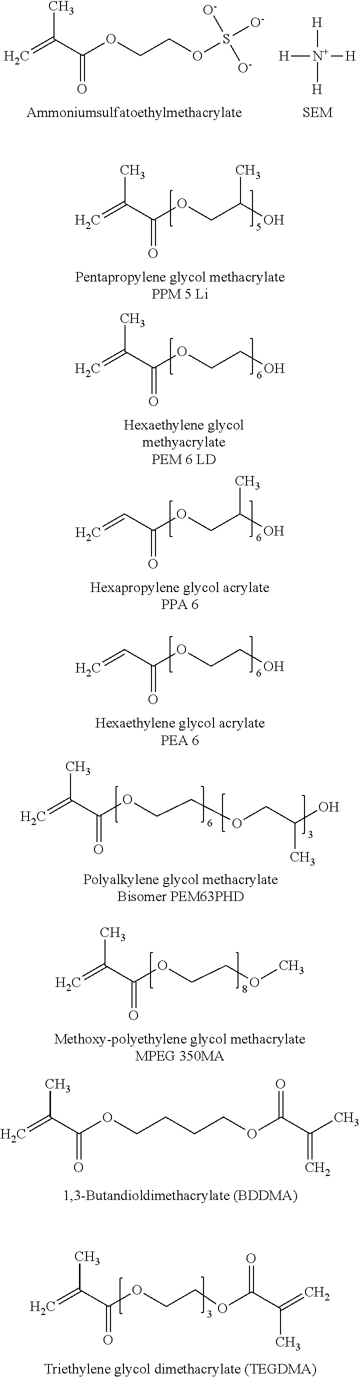

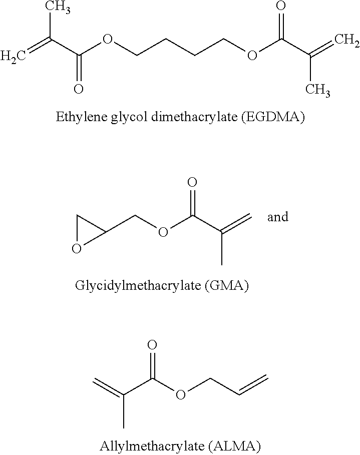

67. Use of an aromatic alcohol for reacting with an aldehyde component which includes at least two C-atoms per molecule, and optionally one (meth)acrylate-polymer for the formation of the capsule walls of microcapsules.

68. Use according to claim 67, wherein formaldehyde-free capsules are formed thereby.

69. A method for the production of microcapsules comprising the steps of:
a) reacting at least one aromatic alcohol, its ether or derivative, and
b) at least one aldehyde component having at least two C-atoms per molecule, and
(c) optionally at least one (meth)acrylate-polymer, from the group of copolymers of 2-acrylamino-2-methyl-propane sulfonic acid or their salts with one or more copolymers from the group of (meth)acrylates, in the presence of a core material, and
d) thereafter hardening the capsules by increasing temperature.

70. A method for the production of a microdispersion comprising microcapsules resulting form the steps of:
a) reacting at least one aromatic alcohol, its ether or derivative, and
b) at least one aldehyde component having at least two C-atoms per molecule, and
(c) optionally at least one (meth)acrylate-polymer, from the group of copolymers of 2-acrylamino-2-methyl-propane sulfonic acid or their salts with one or more copolymers from the group of (meth)acrylates, in the presence of a core material, and
d) thereafter hardening of the capsules is carried out by increasing temperature.

71. The method according to claim 70, wherein the pH-value is increased during the course of the method.

72. The method for the production of microcapsules or microcapsule dispersions according to claim 70, wherein,
a) the at least one aromatic alcohol, the at least one aldehyde component and optionally the at least one (meth)acrylate-polymer, and at least one core material are mixed together at a temperature from 40° C. to 65° C. and a pH-value between 6 and 9,
b) thereafter, at a temperature from 40° C. to 65° C., the pH-value is raised to above 9, and
c) thereafter, hardening the capsules through increasing the temperature to 60° C. to 110° C.

73. The method for the production of microcapsules according to claim 72, further comprising controlling alkalinity of the mixture by an alkaline salt.

74. A method using the microcapsules according to claim 41, comprising the steps of:
liberating active ingredients from the group consisting of fragrances, latent heat storing agents, solvents, catalysts, coating systems, reactive (meth)acrylates, ene-components, anti-microbial agents, greasing agents, lubricants, pharmaceutical agents, cosmetic agents, self-healing agents, waxes, pesticides, fungicides herbicides and insecticides.
